(19) United States
(12) Patent Application Publication
(10) Pub. No.: US 2011/0086368 A1
(43) Pub. Date: Apr. 14, 2011

(54) METHOD FOR IMMUNE RESPONSE DETECTION

(75) Inventors: WAN Y. SHIH, BRYN MAWR, PA (US); JOSEPH CAPOBIANCO, SICKLERVILLE, NJ (US); WEI-HENG SHIH, BRYN MAWR, PA (US); HOSSEIN BORGHAEI, KING OF PRUSSIA, PA (US)

(73) Assignee: DREXEL UNIVERSITY, PHILADELPHIA, PA (US)

(21) Appl. No.: 12/900,137

(22) Filed: Oct. 7, 2010

Related U.S. Application Data
(60) Provisional application No. 61/249,950, filed on Oct. 8, 2009.

(51) Int. Cl. G01N 33/53; G01N 33/573; B82Y 15/00

(52) U.S. Cl. 435/7.24; 436/501; 435/7.21; 435/7.1; 977/920; 977/774

(57) ABSTRACT

A method for detecting one or more immunological response factors that is expressed in response to a therapeutic treatment and/or disease using a piezoelectric microcantilever sensor (PEMS) to assess a patient's immunological response. The method involves measuring a frequency shift of the PEMS caused by binding the immunological response factors to one or more receptors on the PEMS. The method may be used to determine the effectiveness of a prescribed therapeutic treatment and/or monitor the progress of a disease.

### CROSS REFERENCE TO RELATED APPLICATIONS

This application is a non-provisional of U.S. Provisional Application No. 61/249,950, filed Oct. 8, 2009, the entirety of which is incorporated herein by reference.

### STATEMENT OF GOVERNMENT INTEREST

This invention was reduced to practice with Government support under Grant No. RO1 EB000720 awarded by the National Institutes of Health; the Government is therefore entitled to certain rights to this invention. Additionally, portion of the research involved in developing this invention was supported by the Commonwealth of Pennsylvania's Ben Franklin Technology Development Authority through the Ben Franklin Technology Partners of Southeastern Pennsylvania as fiscal agents for the Nanotechnology Institute.

### BACKGROUND OF THE INVENTION

1. Field of the Invention

The invention relates to methods and systems for quantitatively assessing a patient's immunological response to an outside stimulus such as a therapeutic treatment and/or disease using a highly sensitive sensor system.

2. Description of the Related Technology

Current methods for immune response detection are limited to performing Enzyme Linked ImmunoSorbent Assays (ELISA). ELISA, however, has inadequate sensitivity and specificity for accurately detecting or measuring antibodies produced by a patient's immune system. Although recent advances in the field of micro-electro-mechanical systems (MEMS) and nano-electro-mechanical systems (NEMS), have produced sensors having improved detection sensitivities, these sensors still lack the sensitivity and specificity necessary to quantatively measure a patient's immune response. Conventional piezoelectric cantilever sensors, for example, have poor piezoelectric properties, characterized by a low −d₃₁ piezoelectric coefficient of less than 20 pm/v and consequently, lack the sensitivity necessary for quantitative measurement as well as for many detection applications, including in-situ biosensing. Furthermore, it is not appreciated in the art to use sensors as a diagnostic tool to quantitatively assess the effectiveness of a prescribed therapeutic treatment or progress of a disease by monitoring a patient's immunological response.

U.S. Patent Application Publication No. 2006/0178841 discloses an implantable or external bioMEMS/NEMS device including a sensor unit for detecting and monitoring proteins, antibodies, antigens and cells. The sensor unit may be constructed as a microcantilever array and is disclosed for use to detect multiplex character autoantibody response in systemic lupus erythematosus, rheumatoid arthritis, or multiple sclerosis. Additionally, the sensor unit is disclosed for use to diagnose immunity to diseases or investigate pharmacokinetics. Notably, the microcantilever sensor is not disclosed as having sufficient detection sensitivity for quantitative measurements.

U.S. Patent Application Publication No. 2007/0116607 discloses an integrated microarray and microfludic system that is said to enable combinatorial detection of bioagents with a nanomole concentration or at single molecule sensitivity. The microsystem may include an array of reactor-coated silicon microcantilevers with an array of paddles mounted on a piezoelectric crystal, which is said to have attogram sensitivity. The system may be converted to an evaluation tool for performing high throughput tests on drugs.

U.S. Pat. No. 6,092,530 discloses the use of an implantable piezoelectric cantilever sensor positioned on an implant that may be used to monitor the accretion of biological material, such as immune response factors in the bloodstream, and monitor a patient's health over time.

There remains a need to develop a method for quantitatively assessing the effectiveness of a therapeutic treatment and/or the progress of a disease by monitoring a patient's immunological response using an accurate, reliable and highly sensitive sensor system.

### SUMMARY OF THE INVENTION

The present invention is directed to a method for detecting an immunological response. The method involves the steps of detecting a specific immunological response factor by measuring a frequency shift of a piezoelectric microcantilever sensor caused by the binding of the specific immunological response factor to receptors immobilized on the microcantilever sensor employed in longitudinal resonance mode.

### BRIEF DESCRIPTION OF THE DRAWINGS

FIG. 1(a) is a cross-sectional view of an exemplary piezoelectric microcantilever sensor in accordance with the present invention.

FIG. 1(b) is a cross-sectional view of another embodiment of the piezoelectric microcantilever sensor.

FIG. 1(c) is a schematic representation of immunological response factor detection using an exemplary sensor system in accordance with the present invention.

FIG. 1(d) is a schematic representation of immunological response factor detection using a sensor coupled to receptor-bound substrates having quantum dots.

FIG. 2(a) is a flow cell system that can be used in conjunction with the piezoelectric microcantilever sensors of the present invention.

FIG. 2(b) is a small portable sensor system capable of working with an eight piezoelectric microcantilever sensor (PEMS) array and powered by a 9-volt battery.

FIG. 3 is a schematic representation illustrating polarization domain switching.

FIG. 4(a) is a schematic diagram illustrating the immune response triggered in the experiment of Example 1.

FIG. 4(b) is a schematic diagram illustrating the binding of the anti-EGFR antibodies on a PEMS.

FIG. 4(c) is a graph of resonance frequency shift due to binding Panitumab as a function of time.

FIG. 4(d) is a calibration curve for Cetuximab.

FIG. 4(e) is a calibration curve for Panitumab.

FIG. 4(f) is a graph of measured and determined Cetuximab concentration.

FIG. 4(g) is a graph of absorbance as a function of dilution for detecting polyclonal goat antibodies using ELISA.

FIG. 4(h) is a graph of resonance frequency shift as a function of dilution for detecting polyclonal goat antibodies using PEMS.

FIG. 5(a) is a graph showing a comparison of the calibration curves for Cetuximab obtained using PEMS and ELISA.

FIG. 5(b) is a graph showing the change in resonance frequency due to binding ABX-EGFR using PEMS and ELISA.

FIGS. 6(a)-6(b) are graphs comparing detection sensitivity using PEMS and ELISA for a first patient.

FIG. 6(c)-6(d) are graphs comparing detection sensitivity using PEMS and ELISA for a second patient.

FIGS. 6(e)-6(f) are graphs comparing detection sensitivity using PEMS and ELISA for a third patient.

FIG. 7(a) is an optical micrograph showing the PEMS.

FIG. 7(b) shows in-air and in-liquid resonance spectra of a PZT/glass PEMS 970 μm long and 580 μm wide with an 1800 μm long glass tip used in the study.

FIG. 7(c) is a graph of resonance frequency shift versus time of the PEMS: in PBS in period I at t=0-15 minutes, scFv immobilization in period II at 15-44 minutes, PBS rinsing in period III at 43-59 minutes, 30 mg/ml BSA blocking in period IV at 59-185 minutes, 10 mg/ml BSA rinsing and Tween20 rinsing in period V, detection in 600 ng/ml Her2 in 1 in 40 diluted serum in period VI at t=185-278 minutes and rinsing in diluted serum in period VII at t=278-300 minutes.

FIG. 7(d) is a graph of phase angle versus frequency at t=5 minutes (in PBS), 50 minutes (after scFv immobilization), 180 minutes (after BSA blocking), and 275 minutes (after Her2 detection).

FIG. 7(e) is a graph of resonance frequency shift versus time of the PEMS in diluted serum spiked with 0 nM (stars), 0.06 nM (squares), 0.6 nM (up triangles), 6 nM (circles), 60 nM (down triangles), and 600 nM (diamonds) solutions of HER2.

### DETAILED DESCRIPTION OF THE PREFERRED EMBODIMENTS

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation. Further, although certain methods are described with reference to certain steps that are presented herein in certain order, in many instances, these steps may be performed in any order as may be appreciated by one skilled in the art, and the methods are not limited to the particular arrangement of steps disclosed herein.

It must be noted that as used herein and in the appended claims, the singular forms “a”, “an”, and “the” include plural references unless the context clearly dictates otherwise. Thus, for example, reference to “a immunological response factor” includes a plurality of immunological response factors and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

For purposes of the present invention, “disease”, as used herein, refers to any physiological or psychological ailment, disorder, impairment or abnormality.

As used herein, “immunological response factor(s)” refers to any biological indicator produced by a patient's immune system that may be used to gauge a patient's immune response to an outside stimulus such as a therapeutic treatment and/or disease. Exemplary immunological response factors may include immune system cells, such as thymus cells (T-cells), mast cells, cytokines, lymphocytes, macrophages, dendritic cells or natural killer cells; antibodies; antigens; antigen presenting cells; MHC molecules or antigens; HLA complexes or antigens: primary mediators, such as biogenic amines, chemotactic mediators, enzymes, or proteoglycans; secondary mediators, such as leukotrienes, prostaglandins, platelet-activating factors, or cytokines; histamines; platelet-activating factors; neutral proteases; chemotactic factors; cell specific receptors; proteins; cavitants; DNA oligonucleotides; or combinations thereof or combinations thereof. In a preferred embodiment, the immunological response factor may be polyclonal antibodies naturally produced by the body or any type of disease specific T-cells.

As used herein, “therapeutic treatment” refers to any herbal, homeopathic, immunotherapy, genetherapy, pharmaceutical, physical therapy, surgical or other medical intervention. Exemplary treatments may include administration of drugs or monoclonal antibodies, surgical removal of tissue, tissue or organ transplants, cancer immunotherapies, chemotherapy or vaccinations.

As used herein, “specimen” refers to any material extracted from, excreted by or otherwise obtained from a patient. Exemplary specimens may include biopsy tissues or biological fluids, such as blood, serum, urine, saliva or mucus.

As used herein, the terms “width extension mode”, “length extension mode” and “thickness extension mode” refer to various modes of use of microcantilever sensors for detection as described in greater detail below. Specifically, these modes refer to the direction of the induced stress relative to the microcantilever sensor.

Additionally, for the purpose of this patent application, “Q” value as used herein is defined as the ratio of the resonance frequency to the resonance peak width at half the peak height.

The present invention is directed to an immunological response detection method using a sensor system 100 including one or more piezoelectric microcantilever sensors 101. Specifically, the method involves: (1) binding one or more immunological response factors to one or more receptors on a piezoelectric microcantilever sensor 101 and (2) detecting and quantifying the immunological response factors by measuring a frequency shift of a resonance frequency of the piezoelectric microcantilever sensor 101 caused by the binding action thereof. The method may be used to accurately, reliably and quantitatively determine the effectiveness of a therapeutic treatment, monitor the progress of a disease or monitor an immunological response of a patient to an outside stimulus.

### I. Sensor System

Sensor system 100 of the present invention may include one or more microcantilever sensors 101 for detecting the presence of and measuring the concentration of one or more immunological response factors. Microcantilever sensor 101 may be any cantilevered sensor having sufficient sensitivity to quantify the concentration of an immune response factor in a specimen to be tested. In an exemplary embodiment, microcantilever sensor 101 may be any microcantilever sensor capable of longitudinal extension resonance mode operation, such as length extension mode operation, width extension mode operation, and thickness extension mode operation, or combinations thereof. Preferably, microcantilever sensor 101 is constructed as a highly sensitive piezoelectric microcantilever sensor (PEMS). In an exemplary embodiment, sensor system 100 may include an array of two or more microcantilever sensors 101. Optionally, sensor system 100 may further include one or more binding substrates 10 and/or quantum dots 17 to enhance detection sensitivity.

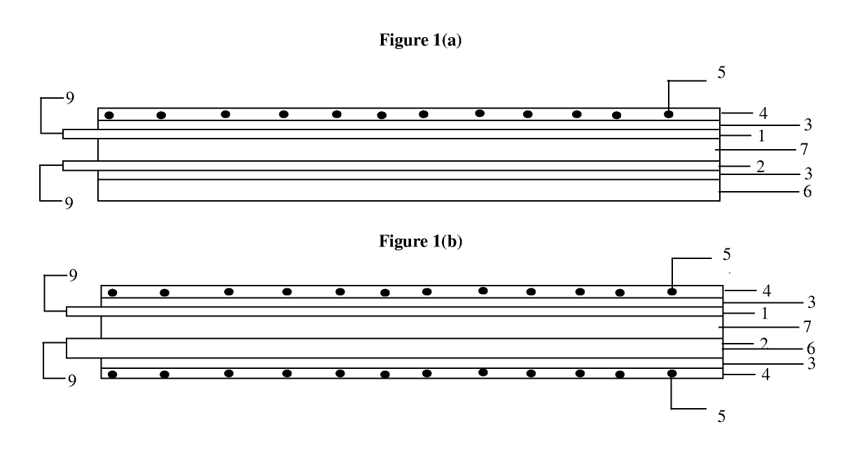

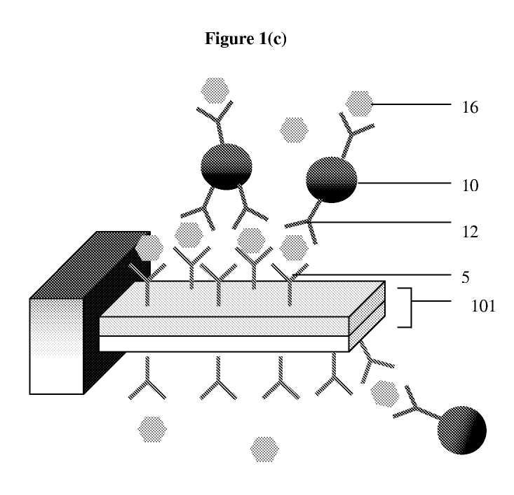

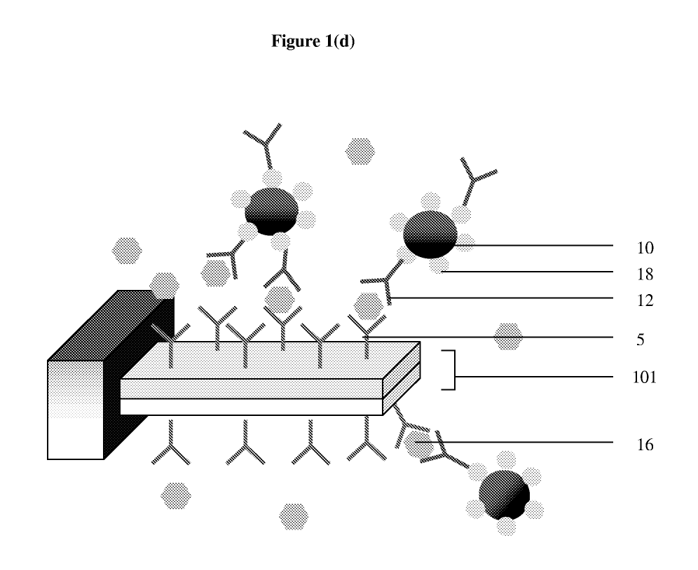

FIG. 1(a) shows the basic structure of an exemplary PEMS, including a first conductive element 1, a second conductive element 2, and a piezoelectric layer 7 positioned therebetween. One or more receptors 5 for binding an immunological response factor may be secured to a surface of the PEMS with an optional receptor immobilization layer 4. Additionally, for applications involving detection in liquid media, the PEMS may further include an electrically insulating layer 3 to increase tolerance of liquid damping or other environmental dampening. The effective mass and/or the effective spring constant of piezoelectric layer 7 changes as a result of binding of an immunological response factor to receptor 5 of the PEMS. By monitoring resonance frequency shifts which result from the mass and/or spring constant changes, the PEMS is capable of rapid, label-free, quantitative, direct, in situ, detection.

FIG. 1(b) is another embodiment of a piezoelectric microcantilever comprising conductive elements 1,2 operatively associated with electrical leads 9, an electrically insulating layer 3, a receptor immobilization layer 4, receptors 5, at least one non-piezoelectric layer 6 and at least one piezoelectric layer 7.

Conductive elements 1,2 may be any element capable of conducting an electrical signal from the piezoelectric layer to a device for detecting that signal. In an exemplary embodiment, conductive elements 1 and 2 are electrodes that may be constructed from any conductive material. Preferably, conductive element 1 is a first electrode constructed from Au/Cr or Pt/Ti and subsequently patterned in several regions. Conductive element 2 is preferably a second electrode constructed from Pt/TiO₂ on SiO₂ for PZT/SiO₂ PEMS or Pt/Ti or Au/Cr on a metal substrate or non-piezoelectric layer and may be subsequently patterned.

A highly piezoelectric layer 7 positioned between conductive elements 1,2, functions to enable electrical detection and actuation within the microcantilever. The piezoelectric layer may function as a driving element, vibrating element, sensing element, or a combination thereof. Applying an AC voltage (input) across piezoelectric layer 7 induces bending and vibration of the PEMS, which in turn induces a piezoelectric voltage that produces readily detectable changes in the magnitude and phase of the output voltage. The resonance frequency of the PEMS may be obtained, for example, by monitoring the maximum of the phase shift of the output voltage. This measurement is accomplished all-electrically, i.e. using both electrical actuation and electrical sensing.

Piezoelectric layer 7 may be constructed from any piezoelectric material, such as (Na_{0.5}K_{0.5})_{0.945}Li_{0.055}Nb_{0.96}Sb_{0.04}O₃ (hereinafter “Sb—NKNLN”), Sb—(Na_{0.5}K_{0.5})NbO₃—LiTaO₃ (hereinafter “Sb—NKNLT”), Sr—(Na_{0.5}K_{0.5})NbO₃—LiTaO₃ (Sr—NKNLN), Sr—Na_{0.5}K_{0.5})NbO₃—LiTaO₃ (Sr—NKNLT), SbSr—(Na_{0.5}K_{0.5}NbO₃—LiTaO₃ (SrSb—NKNLN), SrSb—Na_{0.5}K_{0.5})NbO₃—LiTaO₃ (SbSr—NKNLT), solid solutions with (Bi_{0.5}K_{0.5})TiO₃, (Bi_{0.5}Na_{0.5})TiO₃, Ba(ZrₓTi₁₋ₓ)O₃, BaTiO₃ (hereinafter “BT”), (Bi_{1/2}K_{1/2})TiO₃ (hereinafter “BKT”), (Bi_{1/2}Na_{1/2})TiO₃ (hereinafter “BNT”), Ba(ZrₓTi₁₋ₓ)O₃ (hereinafter “BZT”), Bi(Zn_{1/2}Ti_{1/2})O₃ (hereinafter “BiZT”), (NaₓK₁₋ₓ)NbO₃ (hereinafter “NKN”), BiScO₃—PbTiO₃ BaTiO₃—(Bi_{1/2}K_{1/2})TiO₃ (hereinafter “BKBT”), (B_{1/2}Na_{1/2})TiO₃—(Bi_{1/2}K_{1/2})TiO₃ (hereinafter “BNKT”), (Bi_{1/2}Na_{1/2})TiO₃—BaTiO₃ (hereinafter “BNBT”), (Bi_{1/2}Na_{1/2})TiO₃—Ba(ZrₓTi₁₋ₓ)O₃ (hereinafter “BNBZT”) and (Bi_{1/2}Na_{1/2})TiO₃—BaTiO₃—(Bi_{1/2}K_{1/2})TiO₃ (hereinafter “BNBK”). In a preferred embodiment, the piezoelectric layer is fabricated from highly piezoelectric lead magnesium niobate-lead titanate films (hereinafter “PMN-PT”), such as (Pb(Mg_{1/3}Nb_{2/3})O₃)₁₋ₓ—(PbTiO₃)ₓ (PMN₁₋ₓ—PT_{X}) films, where 0.3<x<0.4, or (Pb(Mg_{1/3}Nb_{2/3})O₃)_{0.65}—(PbTiO₃)_{0.35} (PMN_{0.65}—PT_{0.35}); sodium potassium niobate-lithium niobate solid solutions (NKN-LN); highly piezoelectric lead zirconate titanate (PZT) films; or high piezoelectric lead-free films. In an exemplary embodiment, piezoelectric layer 7 may be fabricated from any highly piezoelectric material with a high −d₃₁ coefficient of about 20 pm/V<-d₃₁<5000 pm/V, preferably about 200 pm/V<-d₃₁<5000 pm/V, more preferably, about 500 pm/V<-d₃₁<5000 pm/V and most preferably, about 2000 pm/V<-d₃₁<5000 pm/V. In another exemplary embodiment, the −d₃₁ coefficient may be greater than about 20×10⁻¹² m/V. Additionally, piezoelectric layer 7 may have a piezoelectric coefficient d₃₃ greater than about 40×10⁻¹² m/V.

Piezoelectric layer 7 may have any structural configuration or dimension. In one exemplary embodiment, piezoelectric layer 7 may be rectangular, triangular, circular, elliptical, or any other geometric shape. In another exemplary embodiment, the piezoelectric layer has a thicknesses of about 0.5 μm to about 250 μm, more preferably, about 0.5 μm to about 127 μm and most preferably, about 0.5 μm to about 100 μm. Piezoelectric layer 7 may further have a length of about 1 μm to about 3 mm and a width of about 1 μm to about 3 mm. In yet another exemplary embodiment, piezoelectric layer 7 may further have a length of about 10 μm to about 3 mm and a width of about 0.5 μm to about 3 mm. Preferably, piezoelectric layer 7 may have a free standing film geometry to enhance domain wall motion and piezoelectric performance.

Optionally, the PEMS may also include at least one non-piezoelectric layer 6, which may be fabricated from any compatible material, including ceramic, polymeric, plastic, metallic material or a combination thereof. In an exemplary embodiment, non-piezoelectric layer 6 may be fabricated from silicon dioxide (SiO₂) or silicon nitride (Si₃N₄) for PZT-thin film based PEMS. In another exemplary embodiment, non-piezoelectric layer 6 may be fabricated from a metal such as Cu, Sn, Ni, Ti, or any combination thereof. Non-piezoelectric layer 6 may also have any structural configuration or dimension. In one exemplary embodiment, non-piezoelectric layer 6 may be rectangular, triangular, circular, elliptical, or any other geometric shape. In another exemplary embodiment, non-piezoelectric layer 6 may have a length of 1 μm to about 3 mm, a width of about 1 μm to about 3 mm and a thickness of about 0.05 μm to about 100 μm.

The PEMS may have a wide variety of structural configurations. A clamp may be used to position and/or attach one or more piezoelectric layers 7 either alone or in combination with one or more optional non-piezoelectric layers 6. In one exemplary embodiment, one or more piezoelectric layers 7 may be bonded to one or more non-piezoelectric layers 6 that are shorter, longer or equal in length thereto. Preferably, one or more non-piezoelectric layer 6 may be shorter than or extend beyond one or more piezoelectric layers 7, so as to form a cantilever tip. When longitudinal bending modes, also known as flexural modes, are not used, the preferred PEMS need not have a non-piezoelectric layer 6 so as to maximize the length extension mode, thickness extension mode or width extension mode resonance frequency shift. In yet another exemplary embodiment, one or more piezoelectric layers 7 may be wider than, narrower than or equal in one or more dimensions with respect to one or more non-piezoelectric layers 6.

One or more receptors 5 may be immobilized onto any surface of the PEMS. Receptor 5 may be any disease specific receptor designed to bind one or more immune response factors naturally produced by a patient's immune system to the PEMS. Furthermore, in order to accurately measure a patient's immune response, receptor 5 is selected so as to specifically bind the species of interest and be incapable of binding a component administered as a therapeutic treatment. For example, receptor 5 may be selected to specifically bind with a polyclonal antibody produced by a patient's immune system in response to intracellular domain antigens released from foreign cells upon being lysed without bonding to administered therapeutic monoclonal antibodies used to bind with an extracellular domain receptor of the foreign cell as part of a therapeutic treatment. Alternatively, receptor 5 may be selected to bind directly to T-cells or any other immunological response factors indicative of a patient's immune response, as described above.

Exemplary receptors, which may be employed as part of the piezoelectric microcantilever, include any receptors, which specifically bind the immunological response factors defined above. In exemplary embodiments, receptor 5 may be disease specific T-cell receptors; antigens for binding antibodies; single chain variable fragments (scFvs) molecules; immune system cells, such as mast cells, cytokines, lymphocytes, macrophages, dendritic cells or natural killer cells; antibodies; antigens; antigen presenting cells; MHC molecules or antigens; HLA complexes or antigens: primary mediators, such as biogenic amines, chemotactic mediators, enzymes, or proteoglycans; secondary mediators, such as leukotrienes, prostaglandins, platelet-activating factors, or cytokines; histamines; platelet-activating factors; neutral proteases; chemotactic factors; cell specific receptors; proteins; cavitants; DNA oligonucleotides; polyclonal antibodies, any type of disease specific thymus cells (T-cells); or combinations thereof.

For example, when assessing the effectiveness of a cancer treatment or the development of cancer in a patient, monomeric and dimeric anti-tumor scFv molecules, such as anti-ECD scFV, which are composed of variable light and heavy chains of antibody molecules that react to cancer markers may be selected as receptor 5. Similarly, when assessing the effectiveness of an anthrax treatment or the development of anthrax in a patient, antibodies specific to Bacillus anthracia (“BA”) spore surface antigens may be selected as receptor 5. When assessing the effectiveness of a tetanus vaccine or the development of tetanus in a patient, human T-cell antigen receptor (TcR) V beta repertoire may be selected as receptor 5 for binding to T-cells induced by the presence of tetanus toxoid.

In an exemplary embodiment, receptors 5 are densely packed on the surfaces of PEMS exposed to the specimen. Preferably, receptors 5 cover the major faces of piezoelectric layer 7 and/or optional non-piezoelectric layer 6.

Any means of adhering receptors 5 to a PEMS surface may be utilized. In a preferred embodiment, receptors 5 may be bound to a surface of the PEMS modified with an immobilization layer 4, such as self assembled monolayers (“SAM”), mercaptopropylsilane (MPS) and bi-functional linkers. In one exemplary embodiment, for purposes of binding scFv, the immobilization coating may be a self assembled monolayer of 3-mercaptoproprionic acid (MPA) on a copper, platinum, or gold-coated electrode activated with 1-ethyl-3-(3-dimethylaminopropy)carbodimide hydrochloride (EDC) and 5 mg/ml N-hydroxysulfosuccinimide (NHS).

For applications involving detection in a liquid, the PEMS may further include an electrically insulating layer 3 in order to electrically separate or buffer conductive element 1 and second conductive element 2, thereby maintaining functionality by preventing conduction. Conductive element 1 may be patterned slightly smaller than the piezoelectric layer 7 to ensure complete insulation of the edges and corners thereof. Any electrically insulating layer 3 may be used as a coating to achieve electrical separation or buffering.

In one embodiment, insulating layer 3 may comprise a 1.5 μm thick parylene (poly-para-xylylene) coating deposited on a conductive element 1,2 by chemical vapor deposition. When placed in static and 1 ml/min flow rate of PBS solution, a parylene insulating layer 3 essentially prevents background resonance frequency shifts greater than 30 Hz and 60 Hz, respectively, over a period of 30 minutes. As a result, insulating layer 3 enables complete submersion of the microcantilever for in situ or in-liquid detection while maintaining a Q value (quality value) greater than about 35.

Alternatively, a PEMS may be insulated using self-assembled monolayers with hydrophobic properties, preferably methyltrimethoxysilane (MTMS) or a combination of MTMS with parylene coatings of varying thicknesses, may also be used. When immersed in a PBS solution, an MTMS insulated piezoelectric microcantilever yields strong resonance peak intensities and prevents background resonance frequency shifts greater than about 30 Hz over a period of 30 minutes.

Other insulation materials may include Al₂O₃, SiO₂ and any functional hydrophobic silane, having a hydrophobic group selected from the group consisting of alkyl, phenyl, alkyl halide, alkene, alkyne, and sulfhydryl. In an exemplary embodiment, the insulation material is mercaptopropylsilane (MPTS), which can also function to immobilize a receptor on the microcantilever.

The resultant PEMS may be capable of electric actuation and electrical detection, chemically inert, thermally stable and is preferably miniaturized to enhance sensitivity. In an exemplary embodiment, the PEMS has a high detection sensitivity of about 1×10⁻¹¹ g/Hz or better, preferably, about 1×10⁻¹¹ g/Hz or better, more preferably, about 1×10⁻¹⁶ g/Hz or better, and more preferably, about 1×10⁻¹⁷ g/Hz or better, and most preferably, about 1×10⁻¹⁹ g/Hz or better. In an exemplary embodiment, the PEMS is designed to detect low levels of circulating proteins in human blood and serum samples and to detect a single cell in solution. These high sensitivities enable early detection of immunological responses, such as antigen specific T-cells and antigen specific auto-antibodies triggered by infections, cancer immunotherapies or vaccination. With such sensitivity, PEMS can be a viable detection tool for monitoring the immunological response of patients undergoing therapeutic treatments, given a vaccine, having allergies or at risk of an infection.

The PEMS may be electrically insulated to enable detection in any sample medium, including air, liquid or solid to enable a variety of biosensing applications. Exemplary PEMS are disclosed in WO Patent Publication Nos. 2008/067386 and 2009/046251 and PCT Patent Application No. PCT/US2009/036048, filed on Mar. 4, 2009, herein incorporated by reference in their entirety. The PEMS may also be incorporated in a portable device and used as a non-invasive means for testing specimens for various pathogens, infectious agents and other markers indicative of disease.

In the exemplary embodiment shown in FIG. 1(c), sensor system 100 may optionally include a plurality of binding substrates 10 that may be placed in a specimen to be analyzed to enhance detection sensitivity. Each binding substrate 10 includes one or more, preferably a plurality of, substrate receptors 12 that are bound to binding substrate 10, and each substrate receptor 12 has one or more binding sites that are specific for binding immunological response factors 16. In one embodiment, substrate receptors 12 are employed to indirectly bind substrate 10 to the PEMS via binding of substrate receptors 12 to immunological response factors 16 that are also bound to receptors 5 bound to a surface of PEMS, as shown in FIG. 1(c). In embodiments where it is desirable to determine the concentration of immunological response factors 16, in addition to being able to detect the presence of immunological response factors 16, it is beneficial to select substrates 10 such that the average number of substrate receptors 12 on each binding substrate 10 is known. It is also advantageous to select substrate receptors 12 and receptors 5 such that the average number of binding sites on each substrate receptor 12 and receptor 5 are known. In this manner, the resonance frequency shift can be mathematically related to the concentration of immunological response factor 16 in the specimen by calibration of the sensor system.

In an embodiment of the invention where it is desirable to measure the concentration of immunological response factor 16, a key aspect of substrate receptors 12 is their ability to selectively bind a specific immunological response factor 16 at a different binding site on immunological response factor 16 than is used to form the bond between immunological response factor 16 and receptors 5. Thus, substrate receptors 12 of binding substrate 10 do not compete with receptors 5 immobilized on a surface of PEMS for binding sites on immunological response factors 16. In one embodiment, each binding substrate 10 includes one substrate receptor 12, in which case target specific receptors 12 function to bind substrates 10 to immunological response factors 16, which will also be bound to receptors 5 on the surface of PEMS. In this manner, the weight of target specific receptors 12 and binding substrate 10 is added to the weight of the bound immunological response factors 16 thereby enhancing the detection sensitivity of the PEMS. Detection sensitivity is enhanced since lower concentrations of immunological response factors 16 can be employed to generate a larger signal from the PEMS due to the added binding stress providing by the additional binding of substrate receptors 12 and binding substrates 10 to the surface of the PEMS. Without wishing to be bound by theory, selecting for micron sized or larger binding substrates 10, the large mass of the bound substrates and stress generated by the bound substrates will dramatically enhance the detection signal even when there are only few immunological response factors on the sensor surface. The micron-size substrate receptors 12 bound to binding substrate 10 are estimated to be able to enhance detection sensitivity by a factor of approximately 10⁶.

Substrate receptors 12 may be any disease specific receptor designed to bind one or more immune response factors naturally produced by a patient's immune system to the PEMS; it is not, however, intended to be capable of binding a material, such as an antibody, administered as a component of a therapeutic treatment to the patient. Exemplary substrate receptors 12 may include the same receptors as described above for receptor 5.

In one embodiment, the substrate receptors 12 are high affinity, high specificity non-competing secondary antibodies which target a specific antigen. A primary antibody located on the surface of a sensor may be used to capture the antigens and subsequently capture any secondary antibody specific receptors which binds to a non-competiting epitope on the antigen.

Secondary antibodies that do not compete with the primary antibodies may be identified from panels of single-chain variable fragment (scFv) antibodies isolated from combinatorial naive phage display libraries or from commercial sources. Additionally, the secondary antibodies may be formulated from new scFv antibodies that are isolated from other scFv phage display libraries in the presence of high concentrations of the primary antibodies to promote the isolation of non-competing clones.

Combinatorial naive phage display libraries are another source for non-competing secondary antibodies. These libraries are typically created through the random combination of human variable light and variable heavy chain domains, resulting in the creation of antibodies that are specific for regions, i.e. epitopes, on target antigens that are not normally immunogenic. The use of phage display therefore significantly increases the areas on the antigen that can be bound by a secondary antibody.

Binding substrate 10 may be any microparticle, more preferably, the substrate is a microsphere, microrod, microplate and most preferably the substrate is a microsphere, microrod or microplate having a diameter of about 0.1 microns to about 100 microns. The microspheres may function like cells or spores that can be captured by immunological response factors 16 attached to the PEMS.

As shown in FIG. 1(d), optionally, binding substrate 10 may be populated with one or more nanoparticles or nanomaterials, such as quantum dots, to enhance the change in resonance frequency of the PEMS. In an exemplary embodiment, the nanoparticles or nanomaterials may be capable of fluorescing, such as fluorescent quantum dots 18, to further enable visualization and imaging of the captured immunological response factors 16. Quantum dots 18 fluoresce under excitation light providing visual or fluorescent verification that the immunological response factors 16 are captured on the PEMS surface and thus confirming the presence of the immunological response factors 16 in a specimen. Therefore, it is possible to view a specimen under a fluorescent microscope and determine the concentration of immunological response factors 16 based on the photoluminescence of the quantum dot 18 populated binding substrates 10. Quantum dots 18 are particularly useful in imaging proteins and cells in biological systems due to their stability against photo-bleaching and their ability to be conjugated to target proteins such as antibodies. Typically, clusters of quantum dots are able to better image biological organisms with brighter luminescence than single quantum dots 18; therefore, quantum dot 18-populated binding substrates 10 are expected to significantly enhance molecular imaging.

Quantum dots 18 may be synthesized using any standard fabrication techniques and may be of any suitable size. The environmentally friendly method for fabricating quantum dots disclosed in W. H. Shih, H. Li, M. Schillo, and W. Y. Shih, “Synthesis of Water Soluble Nanocrystalline Quantum Dots and Uses Thereof,” U.S. Pat. No. 7,335,345, Feb. 26, 2008, is herein incorporated by reference. In addition, nontoxic QDs disclosed in U.S. patent application Ser. No. 11/943,790, “Synthesis of Water Soluble Nanocrystalline ZnS Quantum Dots and Uses Thereof,” filed on Nov. 21, 2007, and near-infrared QDs disclosed in U.S. provisional patent application No. 61/046,899, “Water-soluble Nanocrystalline Quantum Dots Capable of Near Infrared Emissions,” filed on, Apr. 22, 2008 are incorporated herein by reference. Copies of these documents are on file at the United States Patent Office at the time of filing of this International application with the United States Patent Office. In a preferred embodiment, microsphere substrates having a diameter of about 0.1 micron-100 microns may be coated with quantum dots having a size ranging from 3 nm-100 nm.

Additionally, to further increase sensitivity and expedite the detection process, the PEMS may be immersed in a flowing solution for in-liquid detection. Sensor system 100 may be constructed as a flow cell system containing one or more PEMS to enable tailored, rapid and simultaneous detection and quantification of immunological response factors.

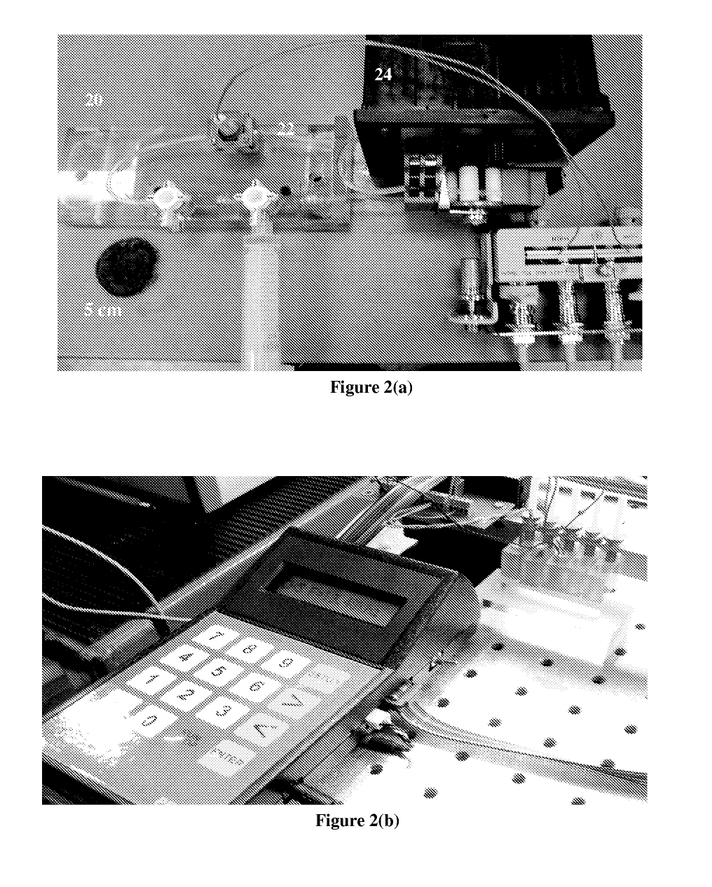

FIG. 2(a) shows a flow cell system 20, with a PEMS holder/measuring unit 22, having a total volume of about 0.03 ml to 10 ml, pump 24, and a mechanism for controlling temperature and humidity (not shown). The flow cell 20 may attain flow rates of 0.01-100 ml/min. The total volume of the flow cell, number of channels and flow rate may vary depending upon the number of compounds to be measured. The flow cell 20 may cooperate with a portable PEMS unit, shown in FIG. 2(b), which has multiple channels for the simultaneous quantification of multiple receptor specific molecules. The portable PEMS is inexpensive and capable of obtaining quick measurements.

Another means for further enhancing sensitivity is by increasing humidity. The mass change per unit area per percent humidity change of PZT PEMS is estimated to be about 1.2×10⁻¹¹ g/Hz/mm²/% humidity. The sensitivity of PMN PEMS by comparison is known to be about three times greater than that of PZT PEMS.

### II Immunological Response Detection Method

The immunological response detection method of the present invention may be used to assess the effectiveness of a therapeutic treatment and/or monitor the progress of a disease.

In general, the immunological response detection method of the present invention involves first selecting one or more receptors 5 that are capable of binding to a specific immunological response factor 16 and immobilizing the selected receptor 5 on a surface of a piezoelectric microcantilever sensor 101. Receptor 5 may be selected to enable detection of early signs of immunological response. For example, receptor 5 may be a receptor for binding a specific antigen-specific T cell and the piezoelectric microcantilever sensor 101 may be fabricated to have a very high sensitivity allowing early detection of low concentrations of the species of interest.

Microcantilever sensor 101 may then be exposed to a specimen. Optionally, one or more binding substrates 10 including one or more optional quantum dots 18 may be introduced to a testing environment containing the PEMS and specimen to be tested in order to enhance detection sensitivity and verify the presence of a select immunological response factor 16. The substrate receptor 12 bound substrates 10 may be introduced to the testing environment or specimen before, at the same time as or after the PEMS is exposed to the specimen. In one embodiment, the specimen and PEMS are first allowed to react for a defined period of time before the substrate receptor 12 bound substrates 10 are introduced to allow the immunological response factors 16 to first bind to the PEMS. This reaction time can vary depending upon the specimen size, immunological response factors 16 and substrate receptors 12. In another embodiment, the specimen and substrate receptors 12 are first mixed to together prior to being exposed to the PEMS.

The presence and concentration level of immunological response factor 16 may then be determined by measuring a change in the resonance frequency of microcantilever sensor 101, induced by the direct binding of one or more immunological response factors 16 to receptor 5.

In operation, an alternating voltage may be applied to conductive element 1 to drive piezoelectric layer 7 of the self-actuating PEMS and a conductive element 2 may be used to detect a shift in the mechanical resonance frequency of the PEMS due to the binding of one or more immunological response factors by the receptors 5. The PEMS is capable of detecting these shifts in resonance frequency by monitoring the i^{th}-mode flexural resonance frequency fᵢ, which is related to the effective spring constant, Kₑ, and effective mass, Mₑ, of the piezoelectric microcantilever at the tip as shown in Equation 1.

$${f}_{i} = \frac{1}{2 π} \sqrt{{K}_{e} / {M}_{e}} \qquad ( Equation 1 )$$

The binding of immunological response factors by the receptors 5 to the microcantilever surface changes the microcantilever mass and the microcantilever spring constant. The resonance frequency shift Δf, expressed in Equation 2,

$$Δ {f}_{i} = {f}_{i} ( - \frac{Δ m}{2 {M}_{e}} + \frac{Δ k}{2 {K}_{e}} ) , \qquad ( Equation 2 )$$

where Δm and Δk denote the mass change and the effective spring constant, model the functionality of the microcantilever.

Due to the presence of the highly piezoelectric layer, the PEMS can exhibit high-frequency nonflexural resonance modes, such as width extension modes, length extension mode and thickness extension modes, that silicon-based microcantilevers lack, as discussed in Q. Zhu, W. Y. Shih, and W.-H. Shih, “Mechanism of the Flexural Resonance Frequency Shift of a Piezoelectric Microcantilever Sensor in a DC Bias Electric Field,” Appl. Phys. Lett. 92, 033503 (2008), herein incorporated by reference in its entirety.

Additionally, as a result of the elastic modulus change mechanism, a PEMS relative resonance frequency shift, Δf/f, was directly proportional to the binding-induced surface stress and inversely proportional to the PEMS thickness where Δf and f denotes a PEMS resonance frequency shift and resonance frequency, respectively. This indicates that under the same detection conditions, Δf could be higher with a high-frequency resonance mode to result in higher detection sensitivity. As non-flexural extension mode resonance occur at a much higher frequency than flexural-mode resonance, detection using non-flexural resonance modes potentially can increase PEMS sensitivity without size reduction. Optionally, during this process, a positive or negative change in the Young's modulus of the piezoelectric layer may be induced, which is preferably a substantial change in the Young's modulus of the piezoelectric layer. In one exemplary embodiment, the change in the Young's modulus may be up to about 70%. The change in the Young's modulus of the piezoelectric layer is preferably greater than about 25%. Most preferably, the change in the Young's modulus may be about 25% to about 70%.

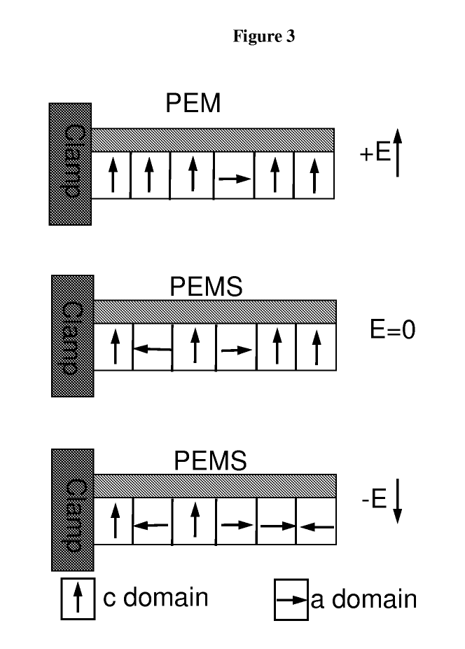

One of the factors that induces a change in the Young's modulus is non-180° polarization domain switching. As shown in the schematic diagram of FIG. 3, by inducing and/or enhancing non-180° polarization domain switching, it may be possible to further increase the detection sensitivity of the PEMS in comparison to non-piezoelectric or weak piezoelectric microcantilevers of the same dimension. One means for inducing non-180° polarization domain switching may be application of stress produced by the binding of immunological response factors. In another exemplary embodiment, non-180° polarization domain switching may be induced by exposing the PEMS to a DC bias electric field. The DC bias electric field may be established using any conventional means and may involve applying a DC voltage across a thickness, length or width of piezoelectric layer 7. Preferably, the established DC bias electric field (E) is from about −20 kV/cm to about 20 kV/cm, more preferably, from about −10 kV/cm to about 10 kV/cm, and, most preferably, from about −8 kV/cm to about 10 kV/cm. A positive value for E denotes an applied electric field that is parallel to the poling direction of the piezoelectric layer. A negative value for E denotes an applied electric field that is opposite to the poling direction of the piezoelectric layer. By establishing a DC bias electric field, the flexural frequency shift and hence, detection sensitivity, may be further increased by a factor of up to about three in comparison to the sensitivity PEMS operated without a DC bias electric field. The DC bias electric field changes the polarization configuration such that it increases polarization domain switching, which in turn enhances the resonance frequency shift enabling enhanced detection sensitivity. The degree of detection sensitivity enhancement is dependent upon the piezoelectric material, the thickness of the piezoelectric layer, whether it is bonded to a non-piezoelectric layer, the physical properties, i.e. thickness and/or material characteristics of the non-piezoelectric layer and any combination thereof.

The method may further involve enabling detection of an immunological response factor using any resonance frequency peak and a longitudinal extension resonance mode. In an exemplary embodiment, the PEMS may be operated in a combination of longitudinal flexural resonance mode and longitudinal extension resonance modes, such as a length extension mode, a width extension mode, a thickness extension mode, or combinations thereof. Preferably, the PEMS may be capable of length extension mode and width extension mode detection, which enables more sensitive detection with high peak frequency intensities and minimized damping effects. In an exemplary embodiment, the PEMS may be used at resonance frequencies within the range of about 10 kHz to about 10 GHz.

When assessing the effectiveness of a therapeutic treatment, the method may involve quantitatively measuring the concentration of one or more immunological response factors 16 prior to administering the therapeutic treatment. In an exemplary embodiment, the concentration may be measured as many times as necessary to establish a pre-treatment baseline, preferably two or more times, more preferably, three or more times. The concentration of the same one or more immunological response factors 16 may be subsequently measured at one or more points in time over the period when the therapeutic treatment is being administered and after the treatment has concluded. The concentration of immunological response factors 16 may be measured periodically throughout the pre-treatment, treatment and post-treatment process and as frequently as necessary to establish statistically significant and reliable results. Subsequently measured concentrations of immunological response factors 16 may then be compared with the concentration levels obtained from the patient at an earlier time. Specifically, the concentration levels measured during or after the completion of treatment may be compared to the patient's pre-treatment levels or previously recorded levels obtained earlier in the treatment or post-treatment process and correlated with known data to assess the patient's condition. Additionally, the measured concentration levels may be compared with established ranges indicative of normal and/or abnormal concentration levels. The trend in the concentration levels of the immunological response factors 16 over time and the comparison with established normal and abnormal concentration ranges may be used to determine the effectiveness of a therapeutic treatment, whether there has been any change in the progress of a disease or condition, or even to provide early detection of potentially dangerous immunological responses to particular treatments such as a severe allergic reaction to a particular therapeutic agent.

Sensor system 100 of the present invention is therefore an effective diagnostic tool that may be used for early detection of diseases, including cancer and infectious diseases, such as HIV. Additionally, it may also be used to qualitatively or quantitatively determine the effectiveness of an administered treatment as well as monitor the progress of the patient's condition throughout treatment. For example, the method may be used to determine a patient's response to and the effectiveness of a vaccine. Specifically, the method of the present application may be used to detect and/or quantify immunological response factors in order to monitor the effectiveness of vaccines and medications, to detect or monitor allergic reactions or to identify abnormalities in a patient's immunological response. In the case of vaccines, the method may be used to determine if, when and how long it takes the administered individual to develop a healthy immune response to a target pathogen and/or early detection of a potentially dangerous allergic reaction to a vaccine. The method would also be useful in studying the immune response to cancer treatments including antibody-based therapies, such as Herceptin, or Cetuximab. Additionally, this approach could be useful in the diagnosis and monitoring of a variety of autoimmune disorders. Sensor system 100 may also monitor the patient's condition in the absence of an administered treatment.

### Example 1 and Comparative Example A

A biological sample was tested for the presence of the immunological response factors Cetuximab and Panitunimab, which are human anti-EGFR antibodies that target the human cell surface receptor EGFR. The concentration of the antibodies was determined based on the measurements obtained from the PEMS and in view of the generated Cetuximab and Panitunimab calibration curves.

In this example, a PEMS was cleaned in piranha solution, diluted 1:40 in DI water. The cantilever was rinsed thoroughly with water and then rinsed twice with ethanol. The cantilever was submerged in a 0.1 mM solution of mercaptopropyltrimethoxysilane (MPS) in ethanol for about 30 minutes. The cantilever was allowed to air dry for about 2 hours and was subsequently rinsed with ethanol. The cantilever was then soaked in a 1% MPS solution in ethanol that was titrated to a pH of about 4.5 using glacial acetic acid for a period of about 36 hours. The cantilever is rinsed with DI water and then with ethanol and dried at 50° C.

The extracellular domain of epidermal growth factor receptor (EGFR-ECD) was first activated with Sulfosuccinimidyl-4-(N-maleimidomethyl)cyclohexane-1-carboxylate (Sulfo-SMCC). For activation a 1 μM solution of antibody was reacted with 80 μM solution of SMCC with a total volume of 300 μl. The solution was gently mixed for about 30 minutes on a nutator (BD company, Clay Adams Brand) at room temperature. Following activation, excess SMCC reagent was removed through filtered centrifugation. During the activation and filtration of the EGFR, the cantilever was soaked in 5 mM ethylenediaminetetraacetic acid in phosphate buffered saline.

The cantilever was then submerged in the 300 μl solution of activated EGFR for a period of about 1 hour to load the EGFR onto the cantilever surface. Afterwards the cantilever was rinsed with PBS buffer and submerged in a 3% BSA solution for about 1.5 hours. Again, the cantilever was gently rinsed with PBS buffer and placed in the 3 ml flow cell. PBS was then circulated through the chamber at a rate of about 0.7 ml/min. This process was continued until a stable baseline was achieved. In general, a stable baseline can be achieved by the drift in resonant frequency is less than 100 Hz over the course of about 1 hour. After obtaining a stable baseline, a 75 μl sample of known antibody concentration (Cetuximab and Panintumab) was spiked into the flow cell.

In order to be systematic, only the first extension mode of vibration in the length direction, i.e. length extension mode, which occurs in the range of 500 kHz to 1 MHz for PEMS were used in the analysis. These length extension mode peaks are different from low-frequency longitudinal bending resonance modes in that the frequency of the length extension mode can be related to the length of the PEMS as F=c/41 where F is the resonance frequency, c the sound velocity, and 1 the length of the cantilever. For the first width extension mode and thickness extension mode, the resonance frequencies are related to the width, w and thickness, t as F=c/2w and F=d/2t, respectively. The difference between an extension mode and a flexural mode is that given the same applied ac voltage, the vibration amplitude of an extension mode is much smaller than that of the flexural mode. For example, give an ac voltage amplitude of 0.1V, the vibration amplitude of the first length extension mode of a 1 mm long PEMS is around one tenth of a nm whereas the vibration amplitude of the first flexural mode is around a hundred nm. Because of the small vibration amplitude involved in a length extension mode, we found that liquid damping does not shift the resonance frequency as much as it does to the low-frequency, high-vibration amplitude of flexural mode, an advantage in in-liquid detection.

As described above, resonant frequency of the first length extension mode can vary with cantilever length, and the detection resonance frequency shift can vary with resonant frequency. Therefore, the shift in frequency was normalized by the peak position (ΔF/F). The resonant frequency, F, was calculated by taking the average of the 20 measured points over the period of the background. The shift in resonant frequency, ΔF, was calculated by averaging the last 20 points of the detection period (t=70 to t=90 minutes) and subtracting this value from the resonant frequency. Spikes in the data, identified as noise, were excluded from the calculations of the shift in resonant frequency.

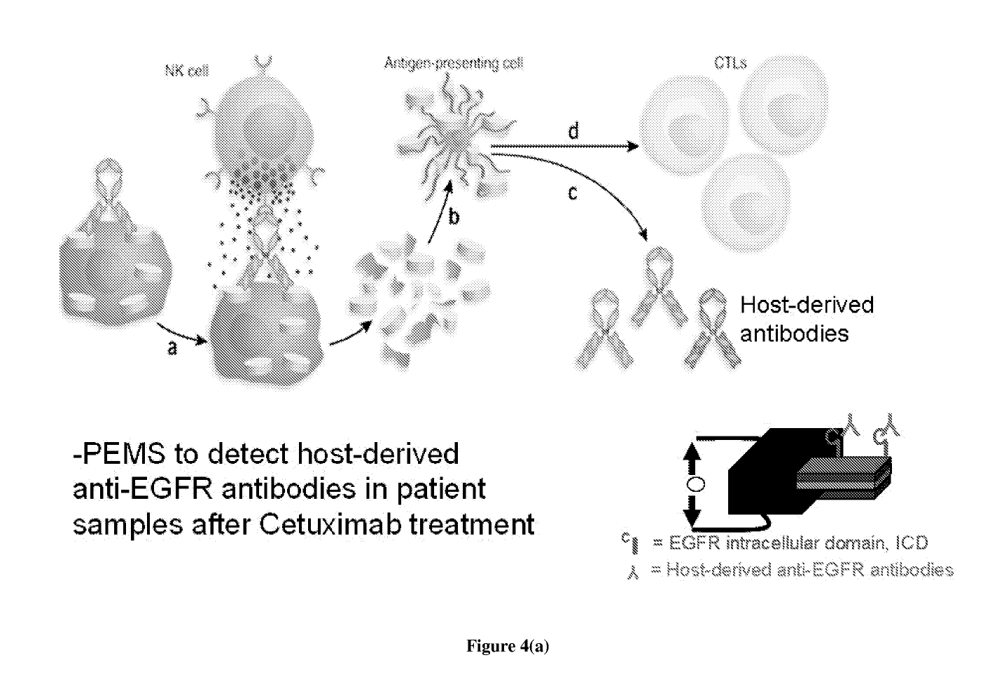

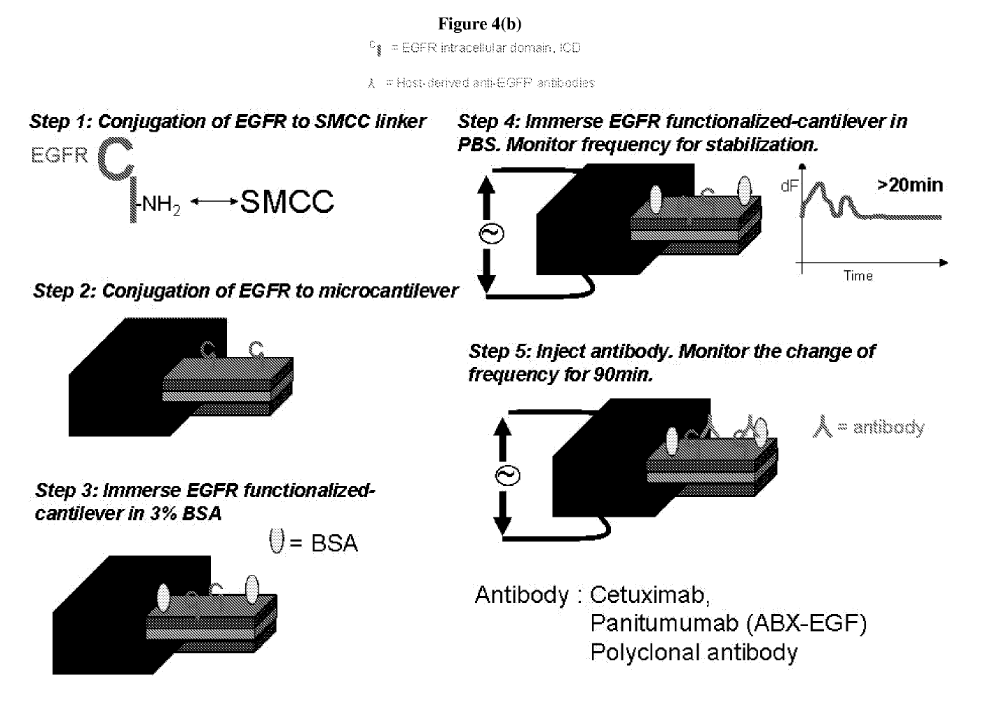

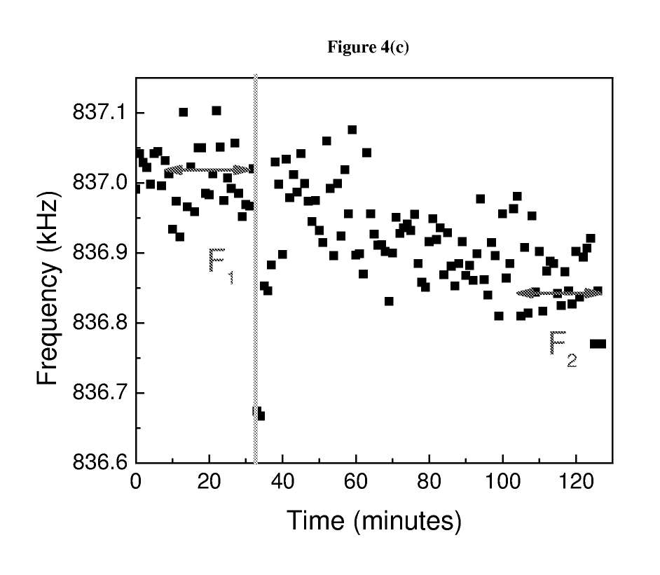

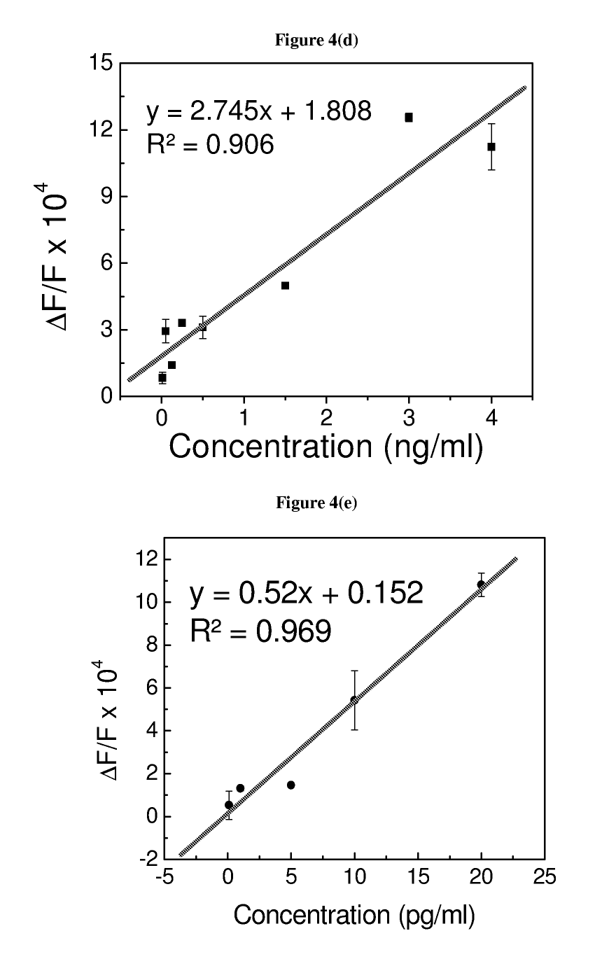

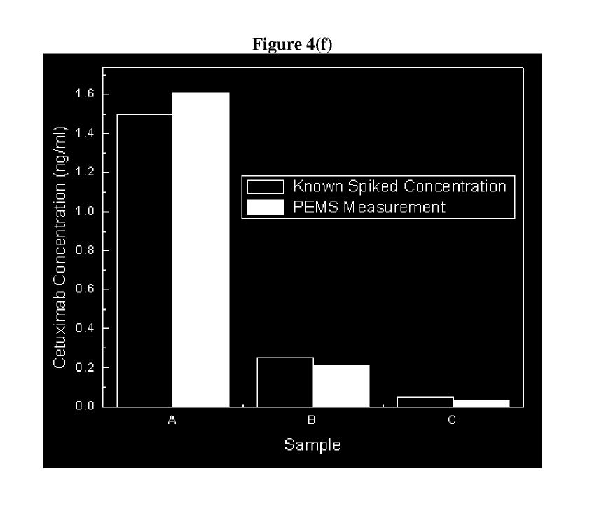

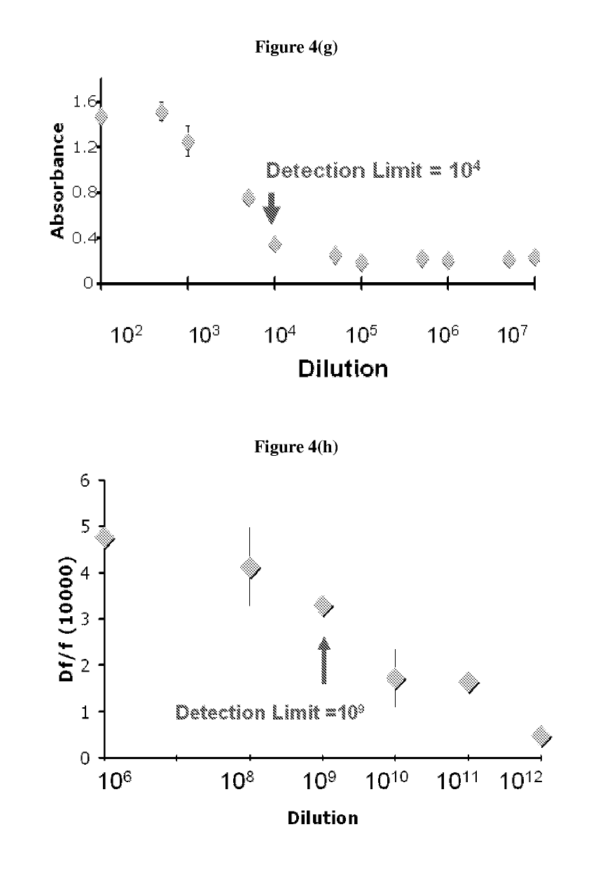

FIG. 4(c) shows the real time response of 5 pg/ml Panitumab in PBS. The length extension mode peak was selected and monitored for about 30 minutes in PBS to ensure a stable background. At t=32 minutes, 75 μl of stock Panitumab was injected into the flow cell to make the final concentration 5 pg/ml. The initial frequency, F₁, was calculated by averaging the points from t=10 minutes to t=30 minutes, yielding a value of about 836,981±50 Hz. Following the injection of Pantiumab, there was a spike in resonance frequency; the six data points following the injection were identified as noise and omitted from the analysis. The value of F₂, was then calculated by averaging from time t=110 to t=130, yielding a value of about 836, 861±50 Hz. The shift in resonant frequency, ΔF=F₂−F₁, was about 120 Hz, which is larger than the standard deviation of the noise, about 50 Hz.

Using a series of concentrations of dilute antibody in PBS, the calibration curves for Cetuximab and Panitumab shown in FIGS. 4(d)-4(e), were generated. The points on these calibration curves were obtained from an average of two separate trials, and the error bars represent the standard deviation between the two trials. For example in the above case, 5 pg/ml panitumab, a value of 1.434×10⁻⁴ is calculated for the normalized frequency shift, ΔF/F. A second trial was conducted where F₁ was about 706,807±40 Hz and F₂ was about 706,702±40 Hz. Using these values, ΔF/F was calculated to be about 1.486×10⁻⁴, and the standard deviation between the two trials was determined to be about 0.036×10⁻⁴.

The calibration curve for Cetixumab was then used to determine the concentration of spiked antibody in the serum, which was diluted 1:40. The resonance frequency was calculated, as discussed above, wherein F₁ was about 568898±30 Hz and F₂ was about 568790±30 Hz. Additionally, the value ΔF/F was calculated to be about 1.898×10⁻⁴, which was then used in a linear interpolation of the calibration curve to yield a concentration of about 33 pg/ml. FIG. 4(f) shows a comparison of concentration of the Cetixumab introduced into the flow cell and the measured Cetixumab concentration using the PEMS.

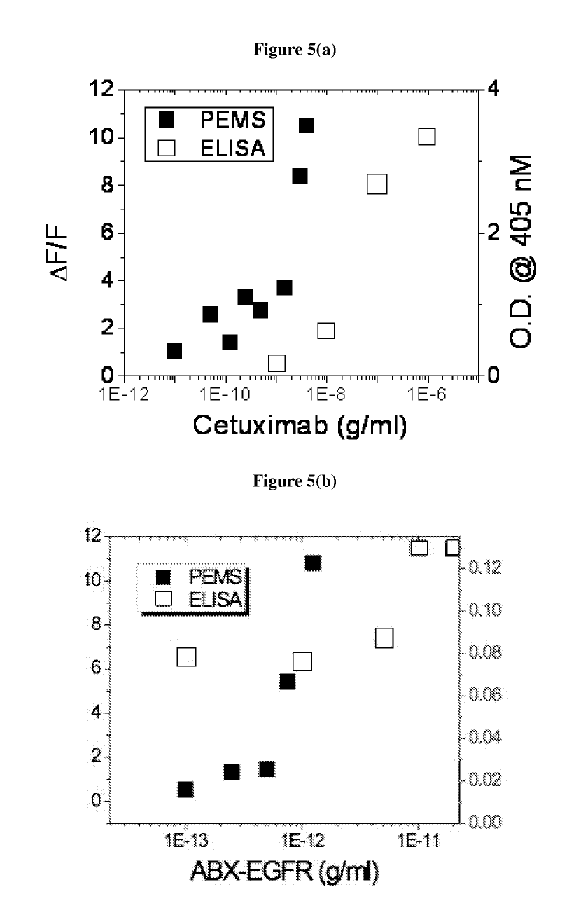

The detection sensitivity of ELISA relative to the PEMS was investigated. FIGS. 5(a)-5(b) show a comparison of an ELISA and PEMS derived calibration curve for Cetixumab and Panitumab, respectively, wherein the left y-axis shows the PEMS response in terms of ΔF/F x 10⁴ and the right y-axis shows the response of the plate reader used in the ELISA protocol in terms of optical density at about 405 nm. FIGS. 5(a)-5(b) show that the limit of Cetixumab and Panitumab detection of the PEMS are about two orders of magnitude better than those achieved by ELISA as ELISA's limitation of detection O.D. was about 0.2.

### Example 2 and Comparative Example B

The ability and detection sensitivity of ELISA in comparison to the PEMS of Example 1 to detect goat polyclonal antibodies was investigated. Biological samples obtained from goats were tested for the presence and concentration of polyclonal antibodies. FIGS. 4(g)-(h) compares the detection sensitivity between PEMS and ELISA in binding goat polyclonal antibodies. These figures demonstrate demonstrating that PEMS has significantly enhanced detection sensitivity.

### Example 3 and Comparative Example C

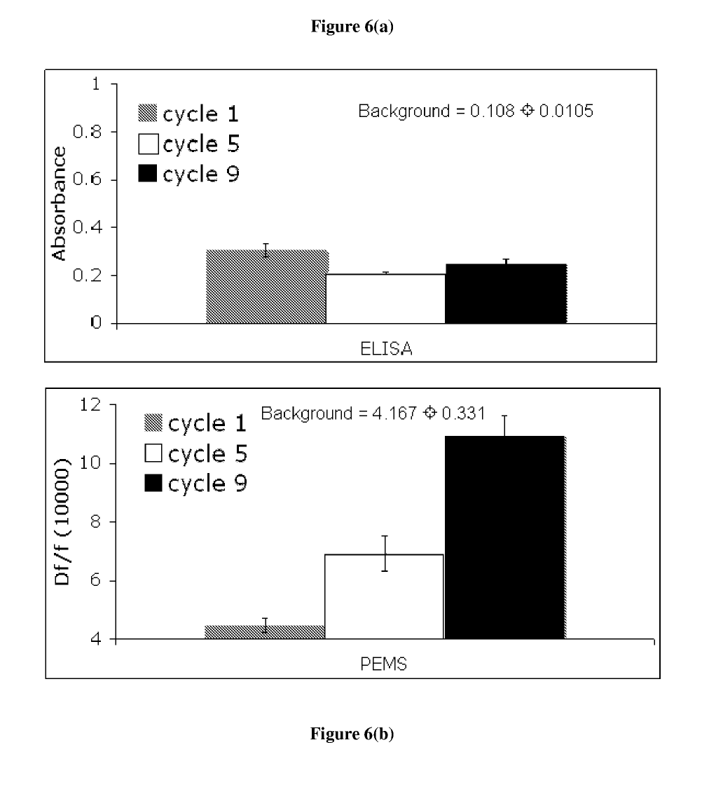

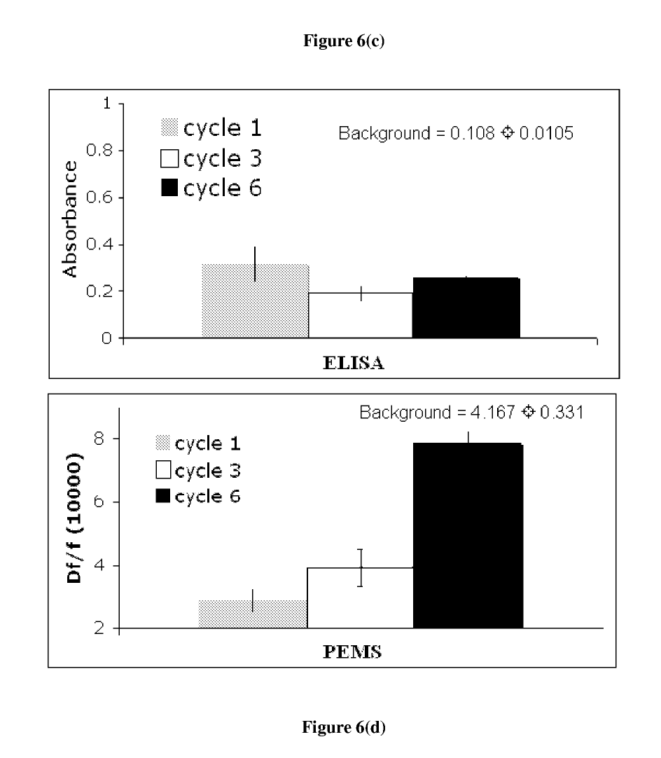

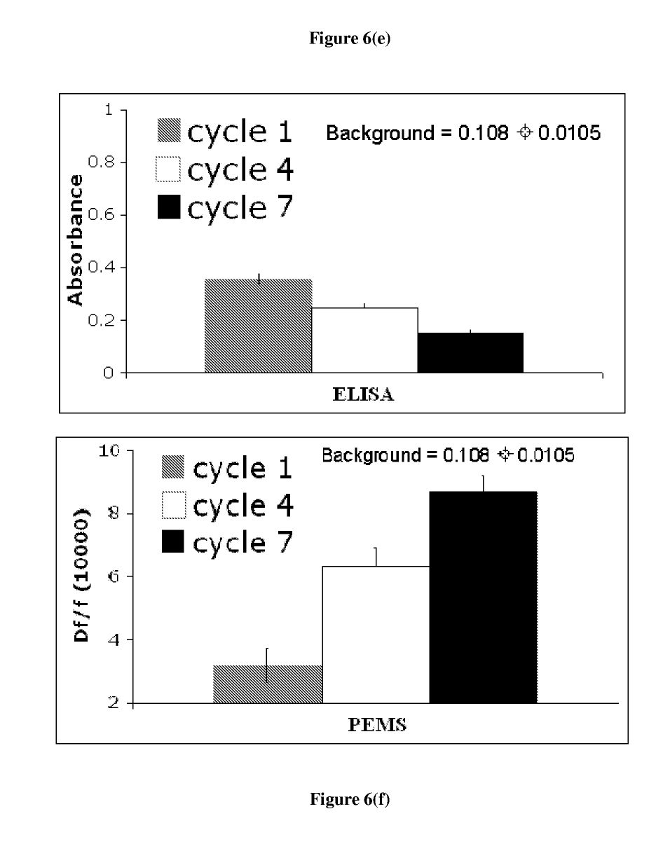

Patient studies were conducted in which Cetixumab was administered to a human in order to treat a tumor. Biological samples obtained from the patients were tested for the presence and concentration of polyclonal antibodies naturally produced by the immune system in response to the presentation of intracellular domain of EGFR. The intracellular domain were produced when a tumor cell was lysed by natural killer cells that are attracted to the tumor cell by therapeutically administered monoclonal anti-EGFR antibody Cetuximab that targets and binds to the extracellular domain of EGFR. FIGS. 4(a)-4(b) are schematic diagrams illustrating the immune response and binding of the anti-EGFR antibodies on the PEMS, respectively. Intracellular domain EGFR receptors were bound to a PEMS to detect the polyclonal antibodies naturally produced by the patient's immune system, host derived specifically anti-EGFR antibodies. FIGS. 6(a)-6(b) shows patient specific responses in which PEMS was found to be substantially more sensitive and capable of detecting the polyclonal anti-EGFR antibodies at substantially lower concentrations than that of ELISA.

### Example 4

A PZT/glass PEMS with H3 scFv receptors was used for real-time, label-free, in situ detection of human epidermal growth factor receptor 2 (Her2) in diluted human serum. Her2 is a cancer antigen that may be an indicator of normal biologic processes, pathogenic processes, or pharmacologic responses to therapeutic intervention. Generally, the Her2/neu proto-oncogene is amplified and/or over-expressed in approximately 20% to about 25% of invasive breast cancers, and the extracellular domain (ECD) of the Her2 protein is often cleaved and released into the circulation with serum concentrations elevated in about 20% to about 50% of patients with primary breast cancer and about 50% to about 62% of metastatic disease. Normal individuals have a Her2 concentration of about 2 to about 15 ng/ml in the blood, whereas breast cancer patients have blood Her2 levels of about 15 to about 75 ng/ml. The present example demonstrates the effectiveness of the PEMS to screen tumor biopsies for Her2 and determine whether a patient is likely to successfully respond to trastuzumab (Herceptin) therapy, a monoclonal anti-Her2 antibody.

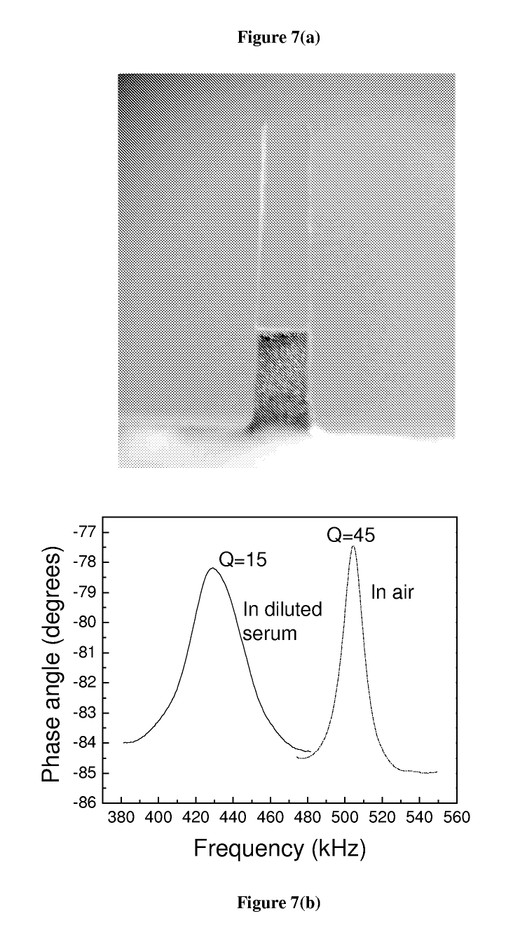

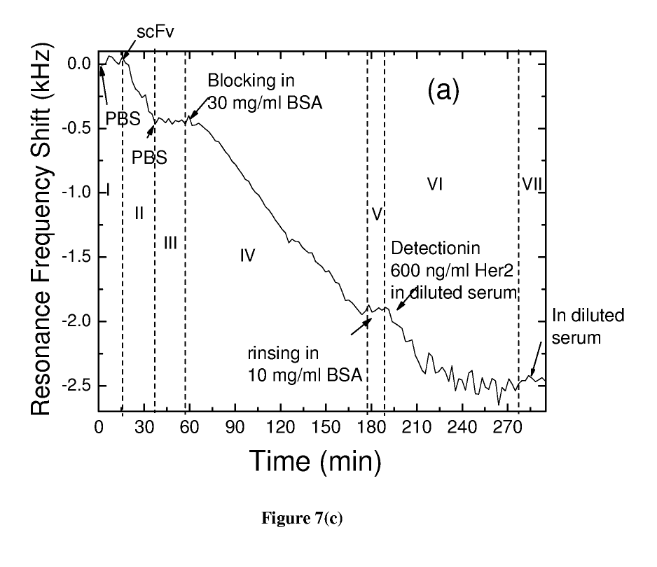

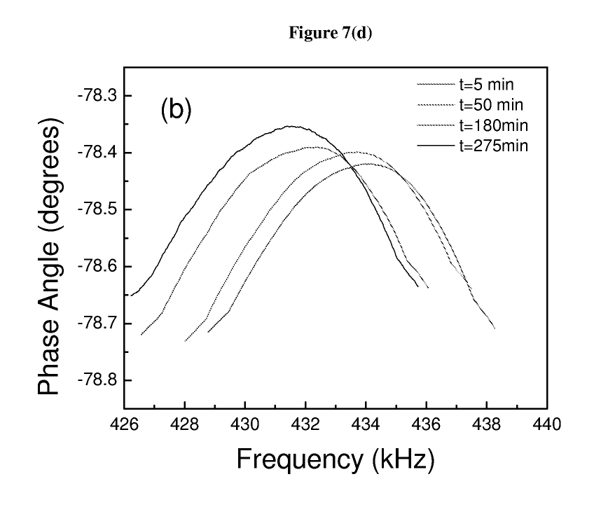

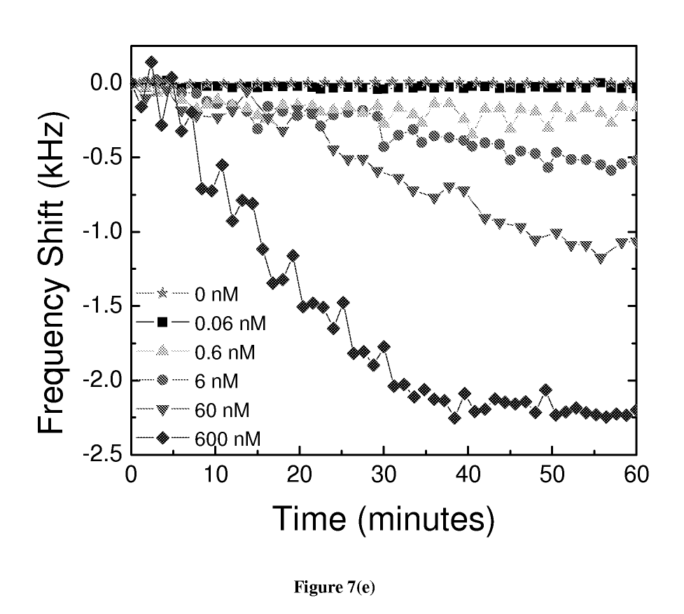

The PZT/glass PEMS used in this study included a commercial PZT layer (T105-H4E-602, Piezo System, Cambridge, Mass.), having a thickness of about 127 μm, a length of about 970 μm, and a width of about 580 μm, bonded to a non-piezoelectric glass layer (Fisher Scientific, Pittsburgh, Pa.), having a thickness of about 75 μm and a glass tip about 1.8 mm in length protruding from a free end of the PEMS. The PEMS was fabricated by first bonding the PZT layer to the glass layer using a nonconductive epoxy (Loctite, Rocky Hill, Conn.) and embedding the PZT/glass bilayer in wax. The PZT/galss bilayer was then cut to strips with a wire saw (Princeton Scientific Precision, Princeton, N.J.). After attaching the wires to the top and bottom electrodes using conductive glue (XCE 3104XL, Emerson and Cuming Company, Billerica, Mass.), a PZT/Glass strip was glued to a glass substrate to form the microcantilever shape. The fabricated PZT/glass PEMS, shown in FIGS. 7(a)-7(b), enables real time, label-free, direct, in situ detection of Her2 in diluted human serum.

To incorporate an insulating MPS layer, the PEMS was first cleaned for about 1 minute in a 20° C. piranha solution, having about two parts of 98% sulfuric acid (Fisher, Fair Lawn, N.J.) to about one part of 30% hydrogen peroxide (FisherBiotech, Fair Lawn, N.J.), that was diluted about 1:100 in water. The PEMS was then soaked for about 4 hr in a container of about 40 mM MPS solution in ethanol that was covered with paraffin film and subsequently rinsing with de-ionized (DI) water. The PEMS was then soaked in a 0.01 M NaOH solution overnight for cross-linking and subsequently soaked in DI water for about 1 hour. The PEMS was then dried overnight in a vacuum-oven (Model 1400E, VWR International) at about 762 mm Hg to conclude the first MPS coating. For each of subsequent MPS depositions, the PEMS was soaked overnight in a freshly prepared 40 mM MPS solution in ethanol titrated to a pH of about 4.5 with acetic acid. This procedure was repeated two times, providing a total of 3 MPS depositions to produce a MPS thickness of about 150 nm.

The Her2 extracellular domain (ECD) was expressed from stably-transected HEK-293 cells and purified using immobilized metal affinity chromatography (IMAC). Anti-Her2 scFv, H3 receptors were isolated from a naïve human scFv phage display library. The Her2 ECD was coated onto a Maxisorp-Immunotube (NUNC, Denmark) at a concentration of about 20 μg/mL in coating buffer (Bup-H carbonate bicarbonate buffer; Pierce) at about 4° C. overnight. scFv-Phage library stock (100 μL; 1.3×10¹³ pfu/mL) was added to the immunotubes to pan, e.g. isolate, anti-Her2 scFv-phage clones. The H3 clones were isolated following four rounds of selection, sequenced and then subcloned into the pCyn expression vector. Soluble scFv were expressed in E. coli TG1, isolated from the periplasmic space and purified by Ni-NTA agarose affinity chromatography and HPLC on a Superdex75 column (Pharmacia). The final yield was about 1-2 mg of pure H3 scFv per liter of expression culture, and specificity for Her2 ECD was confirmed by surface plasmon resonance on a BIAcore 1000 instrument and by flow cytometry against Her2 over expressing human tumor cell lines.

Sulfosuccinimidyl-4-(N-maleimidomethyl)cyclohexane-1-carboxylate (sulfo-SMCC) (Pierce) was used as the bi-functional linker for scFv immobilization on MPS. First, the scFv was linked to sulfo-SMCC using a 1 ml solution 900 nM scFv and 80 μM sulfo-SMCC for 2 hr at 4° C. The NHS-ester in the sulfo-SMCC reacted with a primary amine of the scFv. Unreacted sulfo-SMCC molecules were then removed by repeating microcentrifugation at 6000 RPM with a 10 kD filter (Millipore) three times. The MPS-coated PEMS was then soaked in the sulfo-SMCC-linked scFv solution with about 5 mM ethylenediaminetetraacetic acid (EDTA) (Pierce) for about 2 hr to immobilize the scFv on the MPS coating surface via the reaction of the maleimide of the sulfo-SMCC with the sulfhydryl of the MPS. The adsorption density, Γ, of the SMCC-linked scFv on the MPS was confirmed using a quartz crystal microbalance to be about 7 ng/mm².

To minimize potential non-specific binding, such as binding with Albumin, in diluted serum, the study was carried out in diluted human serum having one part of human serum to 40 parts of PBS. Additionally, the PEMS surface was blocked with a 30 mg/ml BSA (Bovine serum Albumin) solution in PBS prior to detection.

For Her2 detection, the scFv-immobilized PEMS was then immersed in a flow cell containing about 6 ml of a biological sample with a peristaltic pump (model 77120-62, Cole-Parmer's Master Flex, Vernon Hills, Ill.) that pumped the biological sample containing Her2 through the flow cell at a flow rate of 0.7 ml/min. To illustrate the real-time nature of the PEMS, after the scFv was chemically bonded to the sulfo-SMCC, the MPS-coated PEMS was then placed in the flow cell and subjected to scFv immobilization, BSA blocking, and Her2 detection wherein, the two faces of the PEMS were positioned tangential to the flow of the biological sample. FIG. 7(c) shows a graph of the resonance frequency shift of the PEMS within flow cell as a function of time; a shift the in the resonance frequency was first observed upon immersing the PEMS in PBS for about 15 minutes (see period I at about t=0 to 15 min) During Period I, the resonance frequency of the PEMS remained at about 0±30 Hz. Subsequently, during Period II at about t=15 to 44 min, the sulfo-SMCC linked scFv was immobilized, and the resonance frequency of the PEMS decreased with time, yielding a resonance frequency shift of about −430 Hz at about t=44 min After scFv immobilization, PBS was then circulated through the flow cell for about 15 minutes during Period III at about t=44 to 59 minutes to remove any unbound scFvs from the flow cell. During this second PBS period, the shift in resonance frequency was about 0±25 Hz, again indicating that the resonance frequency of PEMS was stable with time in PBS. During period IV at about t=59 minutes to 185 minutes, a 30 mg/ml BSA solution in PBS was circulated in the flow cell to preemptively saturate the non-specific binding of BSA on the sensor surface and minimize potential non-specific binding of Her2 detection in diluted human serum. The resonance frequency shift due to the nonspecific BSA binding reached saturation at about t=172 min, yielding a net resonance frequency shift of about −1470 Hz. Following the BSA blocking, the PEMS was rinsed with a 10 mg/ml BSA and 0.1% Tween20 solution during period V at about t=185 to 195 minutes. Again, during this rinsing period, the resonance frequency of the PEMS remained fairly stable throughout. During period VI at about t=185 to 278 minutes, diluted human serum containing 600 ng/ml of Her2 was introduced into the flow cell and exposed to the PEMS, over which time the PEMS exhibited a resonance frequency shift of about −520 Hz. A final background check of flow of diluted serum was conducted during Period VII at about t=278 to 295 minutes. As shown in Period VII of FIG. 7(c), the resonance frequency of the PEMS also remained stable in diluted serum after detection. To confirm that the resonance frequency shifts shown in FIG. 7(c) were reliable measurements, FIG. 7(d) is a graph of the phase angle as a function of the frequency resonance spectra of the PEMS at about t=5 minutes in PBS, at about t=50 minutes after the scFv immobilization, at about t=180 minutes after the BSA blocking, and at about t=275 minutes after the Her2 detection corresponding to FIG. 7(c). As can be seen, the shape and height of the resonance peak remained roughly constant, indicating that the resonance frequency shifts shown in FIG. 7(c) are reliable. Throughout the detection period shown in FIG. 7(d), the shape of the resonance peak and the Q value remained constant. The longitudinal extension resonance peak the PEMS was therefore used to detect Her2 at a concentration of about 600 ng/ml, which is far lower than the concentration limit of a PZT/glass PEMS when used with general flexural mode resonance peaks were used.

The detection concentration limit of the longitudinal extension mode of the PZT/glass PEMS was determined by examining the dose response of Her2 in diluted human serum. Before each detection, the PEMS was stripped of the bound scFv and reinsulated with MPS. Fresh scFv was then immobilized on the PEMS surface. A PBS was then flowed across the PEMS surface for about 10 minutes to establish the background followed by 30 mg/ml BSA blocking in a flow until the resonance frequency of the PEMS was saturated, which took about 2 hours. The PEMS was then rinsed with 10 mg/ml BSA and 0.1% Tween 20. Subsequently, the PEMS was exposed to diluted human serum spiked with Her2 in one of the following concentrations: 60,000, 6,000, 600, 60, and 6 ng/ml. The scFv was re-immobilize after each detection as opposed to simply releasing the Her2 in a glycine/HCl buffer solution after detection to ensure that the binding performance of the scFv in each detection was roughly identical. FIG. 7(e) shows the resonance frequency shift as a function of time for the diluted serum with 60,000, 6,000, 600, 60, 6, and 0 ng/ml, corresponding to 600, 60, 6, 0.6, 0.06, and 0 nM of Her2 respectively, wherein each curve was the average of two independent tests. At about t=60 min, the PEMS yielded resonance frequency shifts of about 2250±120, about −1060±170, about −531±110, about −160±150, about −35±15, and about 0±15 Hz in diluted serum with 60,000, 6,000, 600, 60, 6 and 0 ng/ml of Her2, respectively. Note that at 0 ng/ml of Her2, the PEMS exhibited no net shift in resonance frequency throughout the 60 minutes exposure period with a standard deviation of about 15 Hz. The about 35±15 Hz shift at about t=60 minutes at 6 ng/ml appeared well above the standard deviation of the control (15 Hz) or that at 6 ng/ml (15 Hz). The reason for the slow, almost linear response over the 60 minutes of detection at this concentration was in part due to the low concentration of 6 ng/ml as well as the moderately low affinity of this scFv. It is also worth noting that the noise level (standard deviation) was considerably higher at a higher Her2 concentration: 120, 170, 110, 150 at 60,000, 6,000 600, 60 ng/ml of Her2, respectively as opposed to 15 Hz at 6 and 0 ng/ml of Her2. The same trend was observed in in-situ detection of other biological systems, indicative that the noise during detection was related to binding, unbinding and re-arrangement of the antigen on the sensor surface.

The study demonstrated that the PEMS may be used for real-time, label-free, in-situ detection of Her2 in diluted serum using the first longitudinal extension mode of a PZT/glass PEMS with H3 scFv immobilized on the MPS insulation layer of the PEMS surface. When operated in the longitudinal extension mode, a PZT/glass PEMS consisting of a 1 mm long and 127 μm thick PZT layer bonded with a 75 mm thick glass layer with a 1.8 mm long glass tip can detect Her2 at a concentration of 6-60 ng/ml (or 0.06-0.6 nM) in diluted human serum, which is about 100 times lower than the concentration limit using the lower-frequency flexural mode of a PZT/glass PEMS of similar dimensions. Additionally, the resonance peak used for the longitudinal extension mode detection was about 504 kHz in air with a Q value of about 45 and about 429 kHz in diluted serum with a Q value of about 15 as shown in the resonance spectra in FIG. 7(b).

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. A method for detecting an immunological response comprising the steps of:
(a) contacting a piezoelectric microcantilever sensor having a receptor immobilized thereon which specifically binds an immunological response factor with a sample of interest,
(b) measuring a resonance frequency shift of the piezoelectric microcantilever sensor caused by binding of said immunological response factor in said sample of interest to said receptor on said piezoelectric microcantilever sensor, and
(c) determining an immunological response from said measured resonance frequency shift.

2. The method of claim 1, wherein during said measuring step, said microcantilever is operated in a resonance mode selected from the group consisting of: longitudinal extension mode, length extension mode, width extension mode and thickness extension mode.

3. The method of claim 1, wherein during said measuring step, said microcantilever is operated in a resonance mode selected from the group consisting of: longitudinal bending mode, longitudinal extension mode, or combinations thereof.

4. The method of claim 2, wherein step (c) comprises the step of determining a concentration of said immunological response factor.

5. The method of claim 4, wherein the method further comprises the steps of:
(d) repeating steps (a)-(c) at least once, and
(e) assessing an effectiveness of a therapeutic treatment based at least in part on said determined concentrations of immunological response factor at different times.

6. The method of claim 5, wherein step (e) comprises the steps of:
(e1) comparing at least two of said determined concentrations of immunological response factor; and
(e2) correlating the result of said comparing step (e1) with the effectiveness of a prescribed therapeutic treatment.

7. The method of claim 6, wherein a first measuring step (b) is carried out prior to administering the therapeutic treatment, and said measuring step (b) is repeated at least once after administering the therapeutic treatment.

8. The method of claim 7, further comprising the steps of:
(f) comparing the determined immunological response factor concentration to a range of normal or abnormal concentrations of the immunological response factor; and
(g) assessing an effectiveness of said therapeutic treatment based at least in part on said comparing step (f).

9. The method of claim 6, wherein said therapeutic treatment comprises administration of a therapeutic agent.

10. The method of claim 9, wherein said therapeutic agent comprises an antibody targeted to an extra-cellular receptor of a cell and said receptor on said piezoelectric microcantilever sensor specifically binds an antibody that binds to an intracellular receptor of a cell.

11. The method of claim 10, wherein the cell is a cancer or pre-cancer cell.

12. The method of claim 3, wherein the method further comprises the steps of:
(d11) repeating steps (a)-(c) at least once, and
(e11) assessing the progress of a disease based on said determined concentrations of immunological response factor at different times.

13. The method of claim 12, wherein step (e11) comprises the steps of:
(e111) comparing at least two of said determined concentrations of immunological response factor; and
(e112) correlating the result of said comparing step (e111) with the progress of said disease.

14. The method of claim 13, wherein a first measuring step (b) is carried out prior to administering the therapeutic treatment, and said measuring step (b) is repeated at least once after administering the therapeutic treatment.

15. The method of claim 14, further comprising the steps of:
(f14) comparing the determined immunological response factor concentration to a range of normal or abnormal concentrations of the immunological response factor; and
(g14) assessing the progress of the disease based at least in part on said comparing step (f14).

16. The method of claim 12 wherein said disease is cancer.

17. The method of claim 2, wherein said receptor is selected to bind an immunological response factor selected from the group consisting of mast cells, thymus cells, cytokines, lymphocytes, macrophages, dendritic cells or natural killer cells; antibodies; antigens; antigen presenting cells; MHC molecules or antigens; HLA complexes or antigens: mediators, chemotactic mediators, enzymes, proteoglycans, prostaglandins, platelet-activating factors, or cytokines; histamines; platelet-activating factors; neutral proteases; chemotactic factors; or combinations thereof.

18. The method of claim 17, wherein said immunological response factor are thymus cells.

19. The method of claim 11 wherein said mediators are selected from the group consisting of biogenic amines and leukotrienes.

20. The method of claim 2, further comprising the step of introducing one or more substrate receptors for binding said receptor on said microcantilever sensor either directly or indirectly to a substrate, said substrate receptors being capable of binding said disease specific immunological response factor, and wherein said substrate receptors do not bind to the same binding site on said disease specific immunological response factor as said receptor on said microcantilever sensor.

21. The method of claim 2, further comprising the step of inducing a change in the Young's modulus of a piezoelectric layer of the piezoelectric microcantilever sensor, and wherein said detecting and quantifying step comprises measuring a frequency shift of said piezoelectric layer having a changed Young's modulus, wherein said frequency shift is caused by binding of said immunological response factor to the receptors on said piezoelectric microcantilever sensor.

22. The method of claim 20, wherein said change in the Young's modulus of the piezoelectric layer is induced by applying a DC bias electric field to said microcantilever sensor to cause non-180° polarization domain switching in said piezoelectric layer.

23. The method of claim 2, wherein said substrate further comprise nanoparticles to enhance the change in resonance frequency.

24. The method of claim 23, wherein said nanoparticles are fluorescent quantum dots and wherein said method further comprises the step of verifying the detection of an immunological response factor in part by observing the presence or absence of a fluorescent signal on the cantilever sensor surface.
